(19) United States
(12) Patent Application Publication
(10) Pub. No.: US 2004/0254501 A1
(43) Pub. Date: Dec. 16, 2004

(54) Achieving a relaxed state

(76) Inventor: James R. Mault, Evergreen, CO (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/344,217

(22) PCT Filed: Aug. 13, 2001
(86) PCT No.: PCT/US01/41689

Related U.S. Application Data
(60) Provisional application No. 60/224,646, filed on Aug. 11, 2000.

(51) Int. Cl. A61B 5/103; A61B 5/117

(52) U.S. Cl. 600/587

(57) ABSTRACT

A process for assisting a person to achieve a relaxed state includes providing a metabolic rate meter, determining the metabolic rate of the person at intervals using the metabolic rate meter, and providing feedback to the person, wherein the feedback is correlated with the metabolic rate of the person, so as to assist the person to achieve a relaxed state.

### FIELD OF THE INVENTION

The invention relates to assisting a person to achieve a relaxed state, in particular to the use of feedback correlated with metabolic rate.

### BACKGROUND OF THE INVENTION

A person's total energy expenditure TEE is the sum of their resting energy expenditure REE and activity energy expenditure AEE, i.e. TEE=AEE+REE.

Resting metabolic rate RMR is sometimes used in place of REE. Conventionally, REE is the energy expended by a person during one day due to resting metabolic processes, and is expressed in units of energy. RMR is the rate of resting energy expenditure, conventionally expressed in units of energy per day. Hence, RMR and REE are often used interchangeably.

A metabolic rate meter, such as an indirect calorimeter, determines the person's metabolic rate, corresponding to TEE. For a person at rest, AEE is zero or close to zero, so that the determined metabolic rate is the resting energy expenditure REE.

Metabolic rate can be determined from a measurement of oxygen consumption and/or carbon dioxide production by a person. The determination of metabolic rate using an indirect calorimeter is described in more detail in U.S. Patents U.S. Pat. Nos. 6,135,107, 5,836,300, 5,179,958, 5,178,155, 5,038,792, 4,917,108 to James R. Mault, M.D.; U.S. patent application Ser. Nos. 09/630,398 and 09/669,125, and published international application Nos. W000/7498, WO01/08554, and WO01/156454 to Mault et al., the contents of all of which are incorporated herein by reference.

Conventional approaches to assisting the relaxation of a person are described, for example, in U.S. Pat. No. 4,124,022 to Gross, U.S. Pat. No. 4,456,347 to Stahly, U.S. Pat. No. 4,632,126 to Aguilar, U.S. Pat. No. 4,777,937 to Rush et al., U.S. Pat. No. 4,819,656 to Spector, U.S. Pat. No. 4,969,867 to Cohen, U.S. Pat. No. 5,081,986 to Cho, U.S. Pat. No. 5,151,080 to Bick, U.S. Pat. No. 5,163,439 to Dardik, U.S. Pat. No. 5,219,322 to Weathers, U.S. Pat. No. 5,266,070 to Hagiwara et al., U.S. Pat. No. 5,289,438 to Gall, U.S. Pat. No. 5,304,112 to Mrklas et al., U.S. Pat. Nos. 5,343,871, 5,465,729, and 5,662,117 to Bittman et al., U.S. Pat. Nos. 5,676,633, 5,681,259 and 6,254,527 to August, U.S. Pat. No. 5,620,463 to Drolet, U.S. Pat. Nos. 5,658,322 and 5,690,692 to Fleming, U.S. Pat. No. 5,720,619 to Fisslinger, U.S. Pat. No. 5,741,217 to Gero, U.S. Pat. No. 6,017,302 to Loos, U.S. Pat. No. 6,024,575 to Ulrich, and U.S. Pat. Nos. 6,026,322 and 6,067,468 to Korenman et al.

An approach to estimating the relaxation state of a person is described in U.S. Pat. No. 4,665,926 to Leuner et al. A physiological state measuring device is disclosed in U.S. Pat. No. 6,081,742 to Amano et al. A respiration training system is described in U.S. Pat. Nos. 4,798,538 to Yagi.

Brain activity control is described in U.S. Pat. No. 4,335,710 to Williamson, U.S. Pat. No. 4,834,701 to Masaki, U.S. Pat. No. 4,883,067 to Knipsel et al., U.S. Pat. No. 5,135,468 to Meissner, U.S. Pat. No. 5,213,562 to Monroe, U.S. Pat. Nos. 5,306,228 and 5,409,445 to Rubins, U.S. Pat. No. 5,352,181 to Davis, U.S. Pat. No. 3,356,368 to Monroe, U.S. Pat. No. 5,899,867 to Collura, U.S. Pat. No. 5,954,630 to Masaki et al., and U.S. Pat. No. 6,097,981 to Freer.

However, these conventional approaches fail to provide methods and systems for providing feedback to a person controlled by a determination of the metabolic rate of the person, so as to assist the person to achieve a relaxed state.

### SUMMARY OF THE INVENTION

Embodiments of the present invention provide methods and systems for assisting a person achieve highly relaxed state using feedback correlated with metabolic rate determined using an indirect calorimeter. A highly relaxed state corresponds to one of deep relaxation where a person's metabolic rate is essentially identical to their resting metabolic rate, or at a very low level. To achieve such a state, AEE has to be reduced to a small value compared with REE.

A process (or method) for assisting a person to achieve a relaxed state comprises providing a metabolic rate meter, determining the metabolic rate of the person using the metabolic rate meter, and providing feedback to the person, the feedback being correlated with the metabolic rate. For example, a process for achieving a relaxed state, comprises breathing through an indirect calorimeter, wherein the indirect calorimeter provides a metabolic rate, and receiving feedback from a feedback device, in communication with the indirect calorimeter, wherein the feedback is correlated with the metabolic rate.

A person breathes through an indirect calorimeter, for example using a respiratory connector such as a mask or mouthpiece. The person receives feedback correlated with the determined value of metabolic rate. Other physiological effects may be correlated with metabolic rate, e.g. breathing volume, breathing frequency, loudness of breathing, pulse rate, skin temperature, skin resistance, blood pressure. These physiological parameters can then be correlated with metabolic rate and used in monitoring the approach to a relaxed state.

The indirect calorimeter monitors VO₂ (oxygen consumption) and/or VCO₂ (carbon dioxide production) and hence metabolic rate as the person breathes. The person's resting VO₂ may already be known, in which case progress to a completely relaxed state may be indicated by a feedback (or biofeedback) mechanism, e.g. a digital or analog display, a changing musical tone, changes in a mellow lighting scheme, gentle swaying of a reed like object, scent production, undulations in the surface on which the person rests, recorded or synthesized voices, etc.

Predictive algorithms may be used to monitor the decline of VO₂ to a RMR baseline level. Once the determined metabolic rate has approached closely the RMR level, the GEM, or accessory device (e.g. a computer in communication with the GEM) indicates this fact, e.g. using audio or visual means. Preferably a loud buzzer is not used as this may disturb the just achieved relaxed state. Audible alarms can be subdued. Visual indication should not disrupt the relaxed state. Other feedback mechanisms may include musical tones, lights (e.g., LED bar graphs), etc. The GEM or an accessory device may contain a speaker and a voice recording, so as to repeat a mantra to the person breathing into it.

Systems according to the present invention can be used to train people in relaxation techniques, to assist the relaxation of animals other than humans, to assist person gain mental control of metabolic processes, and to assist a person increase their metabolic rate.

The following U.S. provisional application is incorporated herein by reference: No. 60/224,646.

### BRIEF DESCRIPTION OF THE FIGURES

FIG. 1 shows a person breathing through a indirect calorimeter, receiving feedback from a feedback mechanism;

FIG. 2 shows a indirect calorimeter having a mask, the indirect calorimeter in communication with an electronic device;

FIG. 3 shows a person on a support, receiving feedback correlated with metabolic rate;

FIG. 4 shows a person provided with a metabolic rate meter and a physiological sensor;

FIG. 5 shows a indirect calorimeter and a physiological sensor in communication with a computing device;

FIG. 6 shows a system by which a person's environment can be modified according to a determined metabolic rate;

FIG. 7 shows a flow pathway for respiratory gases having a pair of ultrasonic transducers, forming part of an improved breathing apparatus according to the present invention;

FIG. 8 shows a patient support system comprising a ventilator, metabolic rate meter, and medication control;

FIG. 9 shows a correlation of a person's metabolic rate with variable environmental factors; and

FIG. 10 shows a system allowing an assistant to monitor and assist at least one subject to achieve a relaxed state.

### DETAILED DESCRIPTION OF THE INVENTION

Relaxation Systems

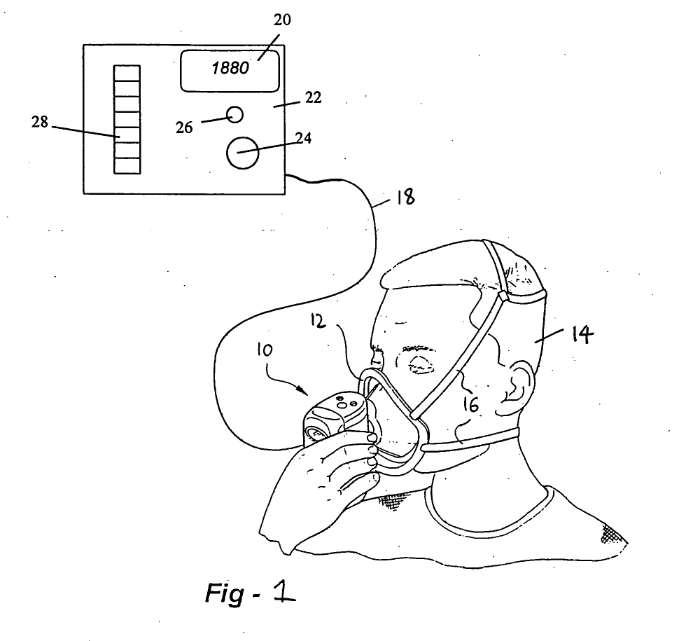

FIG. 1 shows a person breathing through an indirect calorimeter 10. The figure shows a respiratory connector, in the form of a mask 12, placed against the face of a person 14, so as to cover the nose and mouth. Straps 16 are used to maintain the mask in position. The indirect calorimeter (or other respiratory analyzer) 10 is connected by a cable 18 to a feedback mechanism 20. The feedback mechanism 20 comprises a numeric display 22, a speaker 24, an indicator light 26, and a bar graph display 28. As the person breathes through the respiratory analyzer, their metabolic rate is determined, for example, by determining their oxygen consumption. The person can view the numeric display, indicator light, and bar graph during the course of the respiratory analysis. Relaxation of the person can be assisted by providing feedback correlated with the person's metabolic rate. As metabolic rate decreases, the display, bar graph illumination, and indicator light status can change in a manner corresponding with the metabolic rate. For example, the numeric display can show current metabolic rate, bar graph segments can be illuminated or extinguished as metabolic rate falls, and the indicator light can be illuminated when a steady state relaxation has been achieved. The cable 18 can be replaced by a wireless connection, for example using the Bluetooth protocol, IEEE 802.11(b) or related protocol, wireless Ethernet, or other wireless communications protocol.

A color transition from a lamp, such as might be achieved using a red light emitting diode (LED) and green LED wired back to back., can be used to provide a non-disturbing application. The emission wavelength of such a visual indicator can be transitioned from red through orange to green, which could be used as a non-distracting indication of the degree of relaxation.

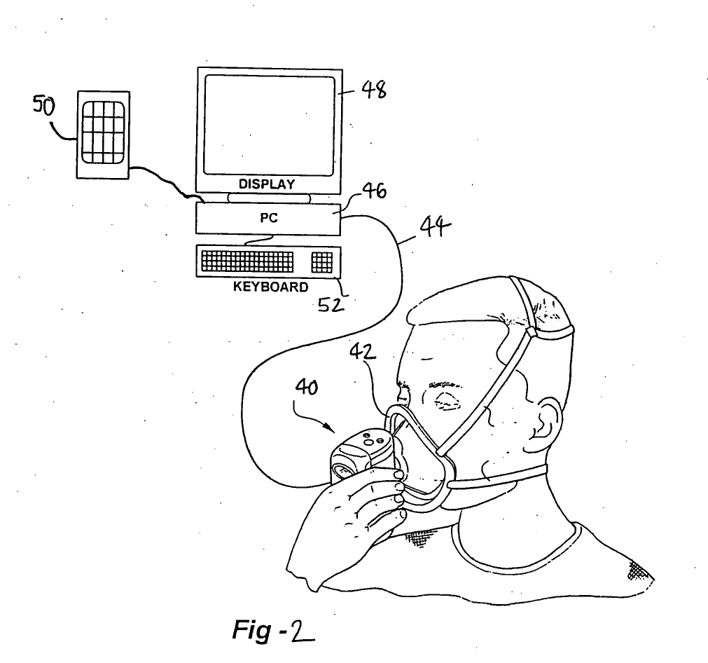

FIG. 2 shows another embodiment in which respiratory analyzer 40 has mask 42 in contact with the face of a user. Data is transmitted over a cable 44 to a an electronic device 46, in this example, a computing device. The electronic device may also be an interactive television, telephone, page, organizer, portable computing device (PDA) or other device. Electronic device 46 has a display 48, speakers 50, and a data entry mechanism (in this example, a keyboard) 52. Other data entry mechanisms include mice, voice recognition systems, touch screens, styluses, touch pads, remote control units, and the like.

In use, the person (or an assistant) uses the data entry mechanism to indicate the start of a relaxation session, metabolic rate measurement session, resting metabolic rate measurement session, breath training session, or other respiratory monitoring session. A bar graph display on display 48 can be used to indicate the decrease in metabolic rate from the start of the session. The higher the bar graph display, the more the metabolic rate of the person has decreased. The scale of the bar chart can be re-adjusted as convenient. A software application program executed by a processor of the electronic device is used to receive metabolic rate measurements received over the cable 44, calculate trends in the metabolic rate data (such as rate of change), calculate absolute changes from a start time of the session, or other reference time, and to generate a visual presentation on the display correlated with the metabolic rate data received from the indirect calorimeter. The software can also be used to determine resting metabolic rate of the person, using metabolic rate data from the indirect calorimeter and determination of when the person is in a resting state, for example when metabolic rate measurements are relatively low and steady state.

In other embodiments, the indirect calorimeter transmits metabolic rate data to a portable computing device, such as a personal digital assistant (PDA) having a display. The display of the PDA can be used to provide visual feedback to the person, for example by displaying a colored bar chart wherein the color and number of illuminated bar chart segments is correlated with the determined metabolic rate of the person. The PDA can further provide an audible signal (such as music, synthesized speech, tones, and the like), or vibrate, so as to provide feedback to the person. A vibrating function of the PDA (or of another device used as a feedback mechanism, such as a wireless phone or pager) can also be used to provide feedback to the person.

An entertainment device, such as a radio, television, computer, web-TV, e-book, Internet appliance, and the like can be used to provide feedback to the person. For example, an indirect calorimeter can transmit a modulated wireless signal to a radio, the radio detecting the wireless signal, and providing an audible signal to the person. For example, a person can listen to an audible signal using the radio, the pitch or other characteristics of the audible signal being correlated with metabolic rate.

The indirect calorimeter can transmit metabolic rate data over a first communications network to a remote computer system, for example using a wireless Internet connection, local wireless network connection, wide area wireless network connection, wireless telephone, cable modem, telephone cable link, or other connection method. The remote computer system can the provide feedback to the person using an entertainment device, the remote computer system and entertainment device being in communication through a second communications network. The second communications network can have a higher bandwidth (or data transmission rate capabilities) than the first communications network. For example, a telephone link, or other low bandwidth data link, can be used to transmit metabolic rate data from the indirect calorimeter to a remote computer system. A high bandwidth connection, such as a cable link, satellite link, optical link, can then be used in the transmission of audio-visual feedback, such as video presentations, or other feedback, to the person over the second communications network. The person receives feedback from the entertainment device while breathing through the indirect calorimeter. The first and second communications networks may also be the same.

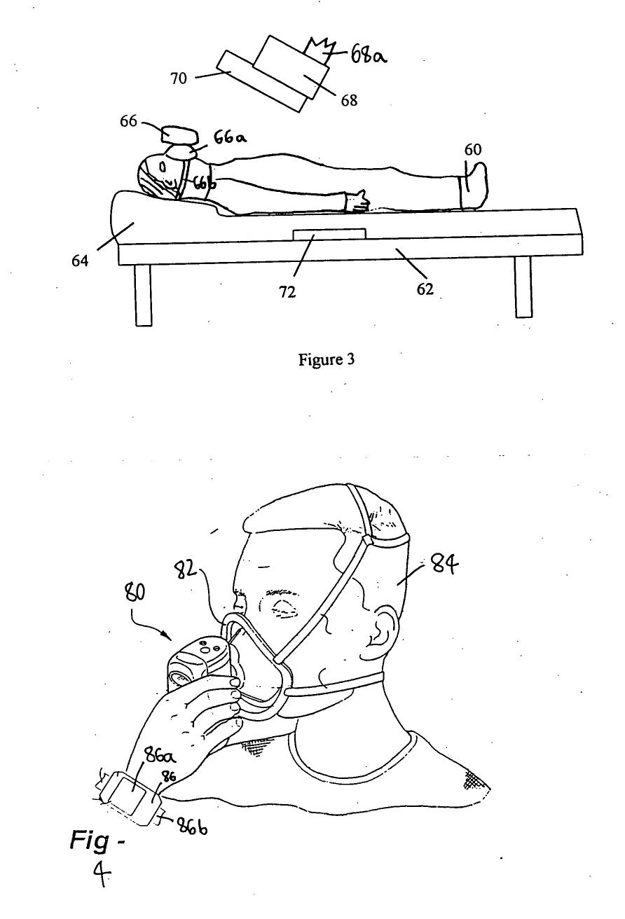

FIG. 3 shows a person 60 lying on a support 62, having cushion 64, breathing through an indirect calorimeter 66, having mask 66a secured by strap 66b. Signals from the respiratory analyzer are transmitted to a control device 68, which modifies the display on a monitor screen 70. The control device and display support, 68a, is not shown in detail, but may for example be an arm which pivots over the person when the person is lying on the support. For example, as the person's metabolic rate decreases, a video image displayed on the monitor screen 70 can be modified to reflect this. A vibrating unit is also provided at 72, so as to apply a mechanical oscillation to the person's body. A vibrating unit can be included in other furniture or support used to support the person during a relaxation process.

FIG. 4 shows a person 84 breathing through an indirect calorimeter 80, having a mask 82 in contact with the face and mouth of the person. A physiological monitor 86 is also provided, which provides a measurement of a physiological parameter which can be correlated with metabolic rate. FIG. 4 shows a wrist-mounted physiological monitor device 86, having display 86a and strap 86b, which can be used to determine a pulse rate for the person. A suitable monitor 86 can use well known photoelectric or oximetry methods to determine pulse rate, and a suitable device can for example be advantageously adapted from the disclosure of U.S. Pat. No. 6,081,742 to Amano et al., incorporated herein by reference. The pulse rate data is transmitted to the indirect calorimeter, for example using a cable or wireless connection. The indirect calorimeter is then used to provide feedback to the person, for example through varying audio tones, spoken commands using synthesized speech, instructions, other noises, and the like, the feedback being correlated with metabolic rate and/or the monitored physiological parameter.

FIG. 5 shows a person 104 breathing through a indirect calorimeter 100 having a mask 102 in contact with the face of the person, secured by strap 102a. The indirect calorimeter 100 is in wireless communication (a cable can also be used with a computing device 106, connected to a display 108 (showing a bar graph 108a with bar heights correlated with determined metabolic rate, though other visual feedback can be provided), a speaker 110 and a data entry mechanism (in this case, a keyboard) 112. The person is further provided with a wrist mounted physiological monitor 114 supported around a wrist by a strap 116. The wrist-mounted device 114 provides one or more physiological parameters, for example pulse rate. The physiological parameter data are wirelessly transmitted to the computing device (a cable can also be used).

Metabolic rate data, and other respiratory parameters such as respired volume, respired frequency, and respiration waveform (the form of the flow rate versus time curve) are transmitted from the indirect calorimeter 100 to the computing device. The computing device is used to provide feedback to the person, through visual presentations such as video, graphic, numeric, or other images on the display 108, or through the generation of audible signals by the speaker 110.

Physiological parameters that can be determined by the wrist-mounted device include pulse rate and skin resistance. Other physiological parameters, which can be monitored and transmitted to the computing device, include brain activity, muscle activity, cardio-vascular parameters, EKGs, and the like. The person's approach to a relaxed state can be monitored through the time dependence of metabolic rate, or the time dependence of one or more physiological parameters. Physiological parameters can also be correlated with metabolic rate, and a correlation relationship stored in a memory of the computing device.

Other metabolic rate meters can be used in place of the indirect calorimeter. The feedback provided to the person can be correlated with the difference between the metabolic rate determined for the person and a resting metabolic rate of the person. The resting metabolic rate can be determined in previous studies, or extrapolated from the time dependence of the determined metabolic rate, or from the time dependence of one or more physiological parameters. The feedback is can also be correlated with a difference between a current metabolic rate determined for the person, and an initial metabolic rate determined for the person at the start of the monitoring process.

The feedback can be correlated with the metabolic rate, for example the pitch, waveform, modulation, component phases, beat frequency component, loudness, or periodicity of one or more components of an audible signal can be correlated with the metabolic rate. Similarly, the characteristics of feedback in the form of a visual presentation on the display can be correlated with the metabolic rate, for example the displayed colors, graphical display, image content, video image content, video play rate, chart, graph, bar-graph, indicator light color, image sharpness, or other property of the visual presentation can be correlated with metabolic rate. Other feedback mechanisms are described elsewhere.

After a physiological parameter is correlated with metabolic rate, the physiological parameter can be used to monitor the approach to a relaxed state, as described in more detail below.

In other embodiments, metabolic rate data from a metabolic rate meter, such as an indirect calorimeter, is transmitted to a set-top box of an interactive television. The set-top box is used to control audio-visual feedback to the person, correlated with the metabolic rate data, For example, a relaxing video with musical accompaniment can be presented to the person using the interactive television, and the tempo of the music slowed as the person's metabolic rate decreases. The luminous intensity, perceived brightness, focus, perceived speed of displayed moving objects of the image can also be changed as the person's metabolic rate decreases.

As is discussed in more detail below, the indirect calorimeter can also be adapted to provide feedback, such as by provision of a tone generator, sound card, scent dispenser, indicator light, and the like.

Correlation of Physiological Parameters with Metabolic Rate

Physiological parameters, such as pulse rate, can be correlated with a person's metabolic rate by simultaneous measurement of the physiological parameter and metabolic rate. For example, metabolic rate can be determined using an indirect calorimeter, and pulse rate can be determined simultaneously using a pulse oximeter. The pulse rate can then be correlated with metabolic rate for the person.

An indirect calorimeter, such as that disclosed in U.S. application Ser. No. 09/630,398, is adapted to determine metabolic rate, and can also be adapted determine and record other respiratory parameters for correlation with metabolic rate. Respiratory parameters, such as respiration volume, respiratory volume, respiration frequency, parameters related to a quotient formed between values related to the inhalation/exhalation duration and values related to the pause between inhalation/exhalation phases (for example, as disclosed in U.S. Pat. No. 4,665,926, incorporated herein by reference), parameters related to exhalation and inhalation durations (for example, a quotient between inhalation duration and exhalation duration), parameters related to a pause duration between the exhalations and inhalations, and other respiratory parameters can then be correlated with metabolic rate using a suitably adapted the indirect calorimeter.

A physiological monitoring device can then be carried, supported, or otherwise associated with the person, the device monitoring one or more physiological parameters (which may include respiratory parameters), and allowing a determination of metabolic rate from a correlation of the physiological parameter(s) with metabolic rate. A suitable device can be advantageously adapted from the disclosure of published Int. App. WO01/26535 to Mault, incorporated herein by reference, and can comprise a physiological sensor, processor, memory, data input mechanism, operational mode selector, real time clock, display, and output devices such as a storage card.

The correlation of a first physiological parameter with metabolic rate may not be very accurate. The value of the first physiological parameter can be mathematically combined with the value of a second physiological parameter to provide a combined physiological parameter, which can then be correlated with metabolic rate. For example, the correlation of pulse rate with metabolic rate may not be precise. In this case, for example, the value of second physiological parameter (such as body temperature, skin resistance, a respiratory parameter, parameter related to brain activity, or a muscle activity parameter) can be combined with the value of pulse rate, so as to form a combined physiological parameter.

Hence, a method for assisting the relaxation of a person comprises: monitoring a physiological parameter for the person; determining a metabolic rate for the person, wherein the metabolic rate is determined using a determined correlation between a physiological parameter value for the person and a metabolic rate value for the person; and providing feedback to the person correlated with the determined metabolic rate of the person. The correlation between the value of one or more physiological parameters and the metabolic rate of the person can be stored in the memory of a feedback device, physiological monitor, on a memory module, or be provided over a communications network.

Feedback can be provided to the person based on a metabolic rate, determined using the correlation between values of one or more physiological parameters and the metabolic rate.

Provision of Relaxing Environment

A system comprising an indirect calorimeter and an environmental control unit, for example, a light control and a heater, can be used to determine the most relaxing conditions for a particular person. Also, feedback correlated with metabolic rate can be provided through the heating or cooling of the environment of the person.

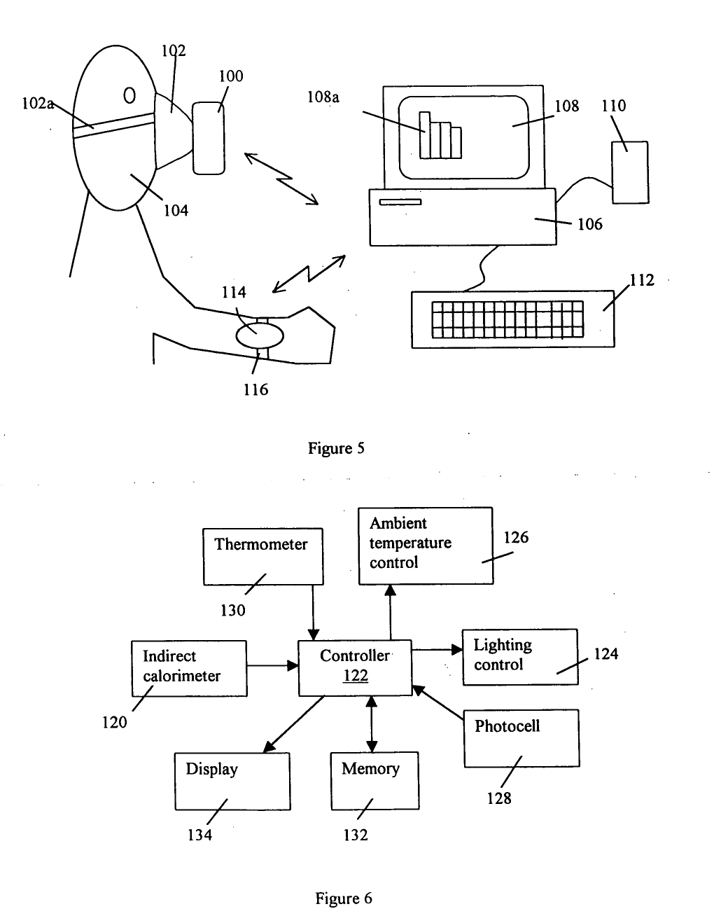

FIG. 6 shows a schematic of a system for determining optimized relaxing conditions for a person, comprising a metabolic rate meter such as an indirect calorimeter 120, a controller 122, a lighting control 124, an ambient temperature control (for example a heating-cooling unit) 126, a photocell 128, a thermometer 130, memory 132, and display 134. The temperature and lighting can be adjusted while metabolic rate is determined, so as to find the optimum temperature and lighting conditions for relaxation of the person.

For example, a light level can be set at a fixed value while the temperature is adjusted over a temperature range. The temperature is then fixed at that value corresponding to the lowest value of metabolic rate determined, while lighting is adjusted over a range. The lighting is then fixed at that value corresponding to lowest metabolic rate, while the temperature is again adjusted. This process can be repeated in an iterative process so as to determine the optimum lighting and heat conditions for relaxation of the person. Other environmental parameters that may be varied include lighting spectrum balance (red/green/blue balance, color temperature), light modulation, background noise level and noise type (such as natural sounds, including wind and water noises, bird calls, human speech, and the like, aroma (aroma type, mixtures, and intensity), mechanical vibrations (such as applied to the person through a chair, bed, or other device), inhaled gas composition (such as ionized content, nitric oxide composition, oxygen concentration, carbon dioxide concentration, or the presence of other vapors), administration of drugs (such as aerosols, injections, rate of administration), meal components (such as the effects of protein, fat, fiber, and carbohydrate content of meals), drink components, displayed images (such as images of people, graphical displays, natural scenes, computer generated graphics), and other parameters. The environmental parameters can also provide feedback, correlated with metabolic rate, to the person, and can be used to determine optimum conditions for measuring resting metabolic rate.

The memory 132 can be used to store determined metabolic rate against one or more variable environmental parameters. The metabolic rate can be presented on the display, for example as a surface contour plot against two variable parameters. This allows a person to easily determine the optimum value of the parameters. A computer software program executed for exampe on a processor within the controller can be used to determine optimized values of variable environmental parameters.

Relaxing conditions can be further correlated with demographic and physiological parameters of the person (for example height, age, weight, ethnicity, body fat percentage, body mass index, resting metabolic rate, personal interests (as determined by questionnaires for example)) so that relaxing conditions determined for one person can be provided for other persons having similar demographics, physiology or interests.

A person can wear an item of clothing comprising a feedback mechanism. For example, a shirt can comprise a heating element controlled according to determined metabolic rate.

Relaxation Methods

Further approaches to assisting a person achieve a relaxed state are discussed.

A method of assisting a person to achieve a relaxed state comprises: providing a metabolic rate meter, such as an indirect calorimeter; determining a metabolic rate of the person using the metabolic rate meter; and providing feedback to the person, wherein the feedback is correlated with the metabolic rate.

Various forms of feedback to the person can be provided. These include visual representations, such as on an electronic display; audio signals; haptic feedback (for example through a body part of the person); mechanical feedback (for example through the motion of supports, furniture, and the like); moving objects (such as swaying reed-like objects); aromas; audiovisual presentations; graphic displays; and the like.

Feedback can be correlated with a determined metabolic rate. As will be described in more detail elsewhere, feedback can also be correlated with the value of physiological parameters which can be, or have been, correlated with metabolic rate, such as pulse rate.

Feedback can be used to provide the person with an indication of their degree of relaxation. Feedback can instead or in addition be used to assist the person achieve a relaxed state.

Visual presentations can include a display of determined metabolic rate, in numeric or graphical forms; bar graphs; colors; graphical shapes; words; and the like.

The characteristics of a displayed image can be modified in a way correlated with determined metabolic rate. For example, the color of an image can shift, for example from red to blue; shapes can change, for example changing number of vertexes of polygons; a bar graph display can illuminate or extinguish segments; and the like.

A person can view the visual representation, and as a result receive feedback as to their degree of relaxation or progress towards a relaxed state. An algorithm can be used to estimate the progress to a relaxed state by extrapolating a falling metabolic rate to a baseline level, then establishing the degree of progress to that baseline level.

Audible signals can also be provided to the person. These include tones, spoken words (for example instructions, mantras, soothing words, recorded speech, speech synthesis), noises simulating natural phenomena such as wind noise, waves, birdcalls, outdoor sounds, other noises which have a relaxing effect on the person, music, crowd noise, and the like.

The characteristics of the audible signal can be modified in a manner correlated with the metabolic rate. For example, the pitch, harmonic content, inherent beat frequencies, waveform, and other parameters of audible tones can be modified. Simulated outdoor noises can become more subdued as progress is made to a relaxed state. For example, using birdcalls, the number of birdcalls can be reduced as the person's state becomes more relaxed. The audible signal can be provided by a source of synthesized speech, so as to enhance relaxation, provide instructions, provide encouragement, and the like. Very low frequency (<50 Hz) sound can be directed at the person, which may have an effect on the relaxation state of the person.

Feedback can also be provided by assuming a person relaxes with time in a predictable manner. In this case, feedback is correlated with the time from the start of a respiratory test or relaxation process. The demographics, physiological parameters, and other parameters may be used to provide an initial estimate of the person's progress towards a relaxed state over time. For example, an initial pulse rate can be used for this estimate.

Feedback can also be provided using mechanical methods. The motion of a moving object, such as a swaying reed-like object or rotating object (such as a mirror ball) can be controlled in a manner correlated with the determined metabolic rate. Vibration of a support for the person, such as a reclining chair, can also be controlled in a similar manner. The feedback can comprise a mechanical oscillation or vibration, with the characteristics of the mechanical oscillation being correlated with the metabolic rate. For example, the amplitude, waveform, frequency, or other property of the oscillation can be correlated with metabolic rate.

Electrical power to an instrument or device can also be controlled in a manner correlated with determined metabolic rate. Instruments and devices can include: evaporative scent dispensers; lamps, such as lava lamps; sound generators; environmental controls (such as air heaters, air coolers, water temperature control (for baths and the like), salinity (for baths and the like); fans; ionizers; coolers; heaters; moving objects, vibrating objects (for example, in beds, recliners, and other support devices), and the like.

The respiratory connector, indirect calorimeter, or accessory device may comprise a scent dispenser so that, for example, indirect calorimetry may be combined with aromatherapy. The feedback can comprises an aroma, with the characteristics of the aroma being correlated with the metabolic rate. For example the intensity of an aroma can be controlled by the heating power of an evaporative scent generator, which can be correlated with the metabolic rate of the person.

Other feedback, which may be correlated with metabolic rate, can include electromagnetic radiation, such as IR, radio waves, and low frequency radiation, which can be directed at the person (for example at the brain of the person), passing of bubbles through a fluid (for example, within a lamp, or through a fluid in which the person is immersed), and controlling the temperature of a bath, tank, or other body (full or partial) immersion equipment.

Indirect calorimetry may be used to quantify the relaxant effect of various aromatherapy treatments. A photo-sensor may also be used to assist in setting subdued light levels to optimum relaxing levels. Indirect calorimetry can be used to quantify the relaxing effects of different lighting levels and colors.

Breathing Apparatus

Embodiments of the present invention can be used with breathing apparatus, such as firefighters' and divers' breathing apparatus, and further with ventilators.

A person in a hazardous environment (such as underwater, in toxic gases, or within a burning building) can experience enhanced oxygen consumption, possibly depleting supplies of breathing gases and hence creating a hazard. The rate of breathing gas consumption, for example from a cylinder, can be determined from the change in cylinder content over time. However, using embodiments of the present invention, metabolic rate can be determined by including ultrasonic transducers within a breathing apparatus. Other embodiments of an indirect calorimeter can also be included within a breathing apparatus, for example indirect calorimeters using different flow sensors.

As described in published Int. App. WO00/07498 to James R. Mault, M.D., respiratory gas analysis (such as oxygen and carbon dioxide concentration of respired gases) can be performed using ultrasound-based gas mass determination, without the need for component gas sensors. (Component gas sensors sensitive to oxygen and/or carbon dioxide can also be used, for example as disclosed in U.S. application Ser. No. 09/630,398, and if present may provide enhanced accuracy). The use of an ultrasound-based analysis of respired gases allows a low cost, portable system to be provided for use in breathing apparatus.

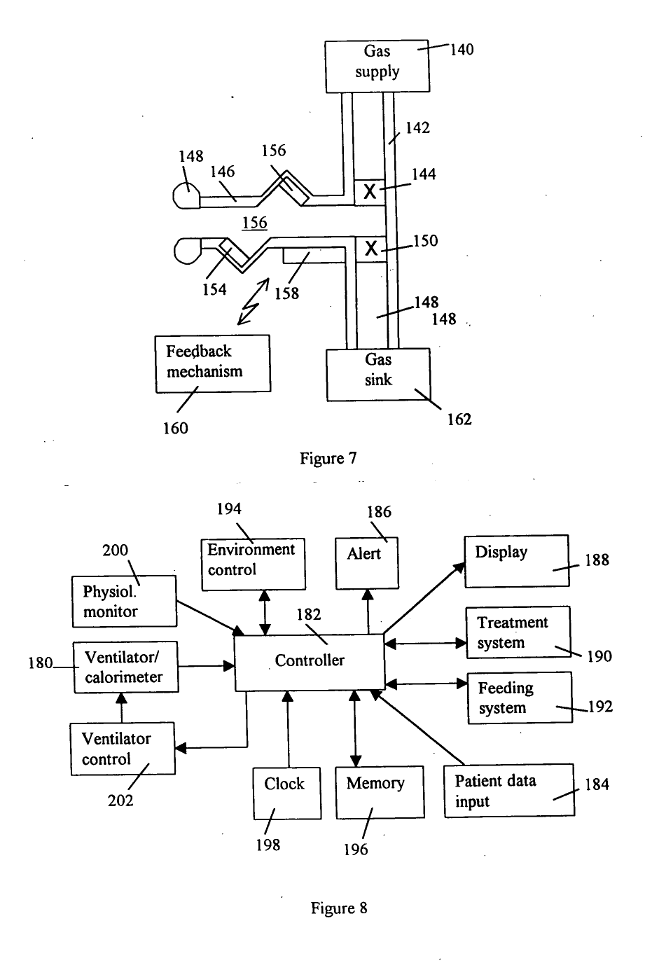

FIG. 7 shows a system forming an improved breathing apparatus, comprising a source of respiratory gases 140, an inlet tube 142, an inlet valve 144, a respiratory tube 146, a respiratory connector 148 (which may be a mask or mouthpiece), an outlet tube 149, an outlet valve 150, a first ultrasonic transducer 152, a second ultrasonic transducer 154, the transducers disposed so as to transmit and receive ultrasonic pulses along a path oblique to the bi-directional flow path of respiratory gases through the respiratory flow path 156 formed by the respiratory tube 146. The system also comprises a feedback mechanism 160 and sink of respiratory gases (such as a port to the atmosphere) 162.

An ultrasonic control system is provided at 158. The control system controls the transducers 152 and 154 so as to determine transmit times of ultrasonic pulses between the transducers. Control systems can be advantageously adapted from those known in the art, for example such as described in U.S. Pat. No. 4,914,959 to Mylvaganam et al., U.S. Pat. No. 5,214,966 to Delsing, U.S. Pat. Nos. 5,419,326, 5,503,151, 5,645,071, and 5,647,370 to Harnoncourt, and U.S. Pat. No. 5,777,238 to Fletcher-Haynes, incorporated herein by reference, and in U.S. patent application Ser. No. 09/630,398 to Mault et al. The control system can further be advantageously adapted to determine respiratory waveform, respiratory frequency and metabolic rate. In other embodiments, the ultrasonic control system contains transducer drivers and circuits for receiving detection signals, but provision of drive commands and analysis of the detection signals are provided by an analysis device in communication (cable or wireless methods) with the ultrasonic control signal.

Metabolic rate can be determined using an ultrasonic gas mass determination, such as described in WO00/07498. Alternatively, the ultrasound transducers are used to determine flow rates, which are integrated with a signal from an oxygen sensor or capnometer exposed to respiratory flow path 156 so as to determine metabolic rate.

The system of FIG. 7 can be adapted to distinguish between high breathing gas consumption (inhaled volume) due to high metabolic rate, and high breathing gas consumption (inhaled volume) due to hyperventilation. A ventilatory equivalent, relating inhaled volumes to oxygen consumption and/or carbon dioxide, can be calculated. Excessive inhaled volumes, for a given oxygen concentration, can be diagnostic of hyperventilation. Excessively high respiratory frequency can also be diagnostic of hyperventilation. If hyperventilation is detected, the carbon dioxide concentration of inhaled gases can be enhanced above atmospheric concentration, using methods know in the art, to prevent depletion of carbonate from the blood of the person. Feedback correlated with ventilatory equivalent can be provided to the person.

The respiratory waveform (flow rate versus time) can be used to determine respiratory problems. For example, if hazardous gases are present in the environment, the effect on a person breathing can be detected and the person warned. The system of FIG. 7 can also be adapted for use in improved ventilator systems. For example, the respiratory connector can be an endotracheal tube.

The determined metabolic rate and other respiratory parameters can be transmitted to the feedback mechanism 160, for example over a cable or using a wireless link. Feedback methods and mechanisms, as described elsewhere in this specification, can be used to assist the relaxation of a person in a hazardous environment. For example, a message can be transmitted to the person suggesting breathing more slowly, indicator lights can indicate a relaxed or stressed state, or other feedback can be provided.

Hence, an improved breathing apparatus comprises a source of inhaled gases (such as a breathing gas cylinder, gas line, the atmosphere (for example in conjunction with a pollutant filter)); a sink for exhaled gases (such as a port to the atmosphere); an indirect calorimeter; and a feedback mechanism, in communication with the indirect calorimeter, which provides feedback to the person correlated with the metabolic rate of the person. The indirect calorimeter can comprise a flow pathway through which exhaled and inhaled gases pass, the flow pathway having fluid coupling with the source of inhaled gas, the sink of exhaled gas, and a respiratory connector through which the person breathes, the indirect calorimeter further comprising a respiratory gas analyzer adapted to determine the flow-rate of gases and the partial pressure of at least one gas passing through at least a part of the flow pathway, the indirect calorimeter further providing a metabolic rate of the person. The respiratory gas analyzer can further comprise an oxygen sensor and/or a capnometer.

A system such as shown in FIG. 7 can be integrated within a helmet, and an audio generator and a display included within the helmet. The helmet can also include telemetry systems, such as a wireless transmitter, so as to transmit metabolic rate, other respiratory parameters, and any other determined physiological parameters to a remote location. A wireless receiver can also be included within the helmet so as to receive feedback and advice from a remote location.

Ventilator

The metabolic rate of a patient on a ventilator can be very high, for example particularly for trauma burn patients. Reducing metabolic rate can be advantageous in assisting recovery of the patient.

Metabolic rate can be reduced by various treatment regimes, such as dispensing medication, application of external treatments such as creams, mists, cooling or heating surrounding air. A metabolic rate meter within a ventilator system can be used to provide data to a feedback mechanism, the feedback mechanism providing feedback to an attendant medical professional. For example, a display can advise a medical professional on suitable treatment regimes for a patient based on a determined value of metabolic rate, along with any other determined parameters.

FIG. 8 shows a patient monitoring system comprising a ventilator 180, the ventilator comprising a metabolic rate meter such as an indirect calorimeter, the ventilator system communicating determined metabolic rate values to a controller 182. The controller 182 can comprise a software program executed by a processor, and may be part of a separate controller module or included within the housing of the ventilator. The system further comprises a data input mechanism such as a keyboard 184, an alert 186, a display 188, a medical treatment application system 190, a patient feeding system such as an infusion pump 192, an environmental control 194, a memory 196, a clock 198, a physiological monitor 200, and a ventilator control 202. The patient's demographic data can be entered using the data input mechanism 184, allowing a normal metabolic rate to be estimated for the patient using for example the Harris Benedict equation or similar equation. Alternatively, a known normal metabolic rate can be entered, for example as determined before an accident, surgery, treatment, or other event responsible for use of the ventilator system. The metabolic rate determined by the indirect calorimeter included in ventilator system 180 can be compared with a known or estimated metabolic rate. Acceptable ratio ranges can be stored in the memory 196. Treatment suggestions can be provided to an attendant medical professional using the display 188, based on the absolute value of metabolic rate or the value of determined metabolic rate with a known or estimated normal metabolic rate. The display 188 can display visual representations of the patient's metabolic rate, such as numeric displays, bar charts, and other graphics such as colored warning symbols. Treatment suggestions and the correlation with metabolic rate can be stored in the memory, for example as part of an expert system. Medical treatment can be dispensed, for example using misters, aerosols (possibly built into the ventilator flow path), sprays, injections, infusers, and other applicators. Feeding can also be controlled by the determined metabolic rate, for example as discussed in Int. App. WO01/156454 to Mault et al. Medication application can be combined with feeding, using an injection mechanism or infusion pump. For example, a nutritional liquid may be combined with drugs and the composition and application of the combination controlled by the determined metabolic rate.

Advice can be provided to an attendant medical professional using the display 188. The alert 186 can sound or illuminate, depending on the nature of the alert, if the metabolic rate is within a dangerous range. Dangerous ranges may be stored within the memory 196. The environmental control 194 can also be controlled in a manner corresponding to the patient condition. For example, the environment can be cooled if the body temperature of the patient is too high. The physiological monitor system 200 can provide any suitable or useful diagnostic physiological parameter, such as pulse rate, body temperature, and other parameters. The physiological monitor can be a single sensor, or a combination of sensors.

An expert system, for example a software programming running within a processor of the controller 182 in conjunction with a database stored within memory 196, can be used to diagnose problems using available data such as metabolic rate, physiological parameters provided by the physiological monitor system 200, other respiratory parameters such as breathing frequency, breathing volume, and the like. The environmental control can also comprise control of ambient light, noise, aroma, and any other environmental factor. This might include the playing of soft music, adjusting lighting, and providing other feedback mechanisms such as aromas as discussed elsewhere in this specification. It can be found that even for an unconscious patient, certain feedback mechanisms will be useful in assisting the recovery of the patient, for example through the lowering of a metabolic rate.

Improved Resting Metabolic Rate Measurements

Determination of resting metabolic rate (RMR) for a person generally assumes that the person is at rest. The relaxation feedback described herein can be used to improve the accuracy of the assumption.

Relaxation encouragement and feedback can be advantageously used to assist a person achieve a relaxed state, so as to increase the accuracy of RMR determinations. Physiological parameters can be monitored during relaxation of the person and used to monitor the approach to a relaxed state. Such physiological parameters include respiratory frequency, pulse rate, core body temperature, respired volume, respiration waveform, brain activity, and the like.

Resting values of physiological parameters can be determined, using for example extended resting metabolic rate tests, in which it is very likely that the person has reached a true relaxed state. These resting values of physiological parameters can then be correlated with a resting metabolic rate. As a determined value of a physiological parameter approaches the value known to be correlated with resting metabolic rate, feedback can be provided to the person to reflect the approach of the monitored physiological parameter to the resting value.

Alternatively, feedback can be provided based on an initial value of metabolic rate; for example, the feedback may be based on the change of a physiological parameter or metabolic rate from an initial value.

Demographic or other data can be used to estimate RMR, and feedback based on a difference between the measured metabolic rate and estimated RMR can be different. However, a problem with this approach is that the estimated RMR can be inaccurate. Feedback can be provided to the person based on a metabolic rate determined from the time from the start of a relaxation process, using the time dependence of a measured metabolic rate for the person determined as relaxation progresses. The time dependence of relaxation can be determined for a person under a number of environmental conditions, and an appropriate time dependence used for the control of feedback.

A person can be determined to be in a fully relaxed state when the measured metabolic rate reaches a steady state value. Feedback can be provided so as to indicate this, as discussed elsewhere in this specification. For example, an indicator light may illuminate at this time.

In other embodiments, sensations are provided to the person, the sensations being chosen so as to correspond to certain sequences, time dependence, content, patterns, or other characteristics, of previous feedback provided to the person. This can be effective if the characteristics of the previous feedback were found to be particularly successful in relaxing the person, as determined using metabolic rate measurements. For example, a sequence of images can be found to be particularly relaxing to a person. Future metabolic rate measurements can be made after presenting a substantially similar sequence of images to the person. A person can also view the images, for example on the display of a PDA, for relaxation purposes, for example before or after stressful events.

In other embodiments, an algorithm is used to determine when a person has achieved a fully relaxed state. In this case, the metabolic rate and monitored physiological parameters will be in a steady state, that is not changing with time. For example, a trend algorithm can be advantageously adapted from Morgan et al., European Pat. App. EP0691631B, U.S. Pat. No. 5,680,310, incorporated herein by reference. When all trend values, or at least one trend value, corresponding to parameters such as metabolic rate, pulse rate, and any other monitored parameters, are below some pre-set value, the person can be considered to be in a fully relaxed state. Other algorithms can be used to determine when metabolic rate is effectively steady state, for example by comparing one or more metabolic rate values separated by some time value(s). Feedback can be provided to the person, the feedback being correlated with the trend of the metabolic rate, and/or the trend in other physiological values. Feedback can also be correlated with higher order derivatives of the time dependence of any monitored parameter.

Calorimeter Modifications

An indirect calorimeter can be provided with an earpiece or headphone socket, so that the person undergoing respiratory analysis and metabolic rate determination can receive spoken advice, suggestions, instructions, music, and other audible signals. These audible signals can be provided by an outside source, such as using wireless transmission from a remote device or assistant, and these signals can also be provided from an internal memory, plug-in memory module, wireless connection to a communications network, and the like.

An indirect calorimeter can also be adapted so as to communicate with an oximeter, other pulse-measuring device, or other physiological sensor, so as to determine the correlation of pulse rate or other physiological parameter with determined metabolic rate. Also, the change in physiological parameter over time can be used to estimate the person's approach to a resting state. For example, if pulse rate suddenly increases, a metabolic rate measurement can be stopped until the pulse rate has fallen again.

An indirect calorimeter can be provided with a headphone socket. A determined metabolic rate for a person can be converted into an audible signal, for example by using an analog voltage correlated with metabolic rate to control a voltage-controlled oscillator. A second oscillation can be provided corresponding to a known, estimated, or predicted resting metabolic rate, so that the person hears a beat frequency of lengthening period as their metabolic rate falls towards a resting value. A digital representation of metabolic rate can also be used to control an audible signal.

Hence, a method for assisting a person to achieve a relaxed state, comprises: providing an indirect calorimeter, wherein the indirect calorimeter comprises a flow pathway and a pair of ultrasonic transducers providing transducer signals correlated with the flow speed of gases through at least a part of the flow pathway; having the person breath through the indirect calorimeter; calculating the metabolic rate of the person using the transducer signals provided by the pair of ultrasonic transducer; transmitting the metabolic rate to a feedback mechanism; and providing feedback using the feedback mechanism, wherein the feedback is correlated with the metabolic rate. The feedback mechanism can be incorporated in or supported by the housing of the indirect calorimeter. Alternatively, the feedback mechanism can be a device in communication with the indirect calorimeter through a cable or wireless link. The respiratory gas analyzer can further comprise an oxygen sensor and/or a capnometer.

Correlation of Metabolic Rate and Environmental Parameters

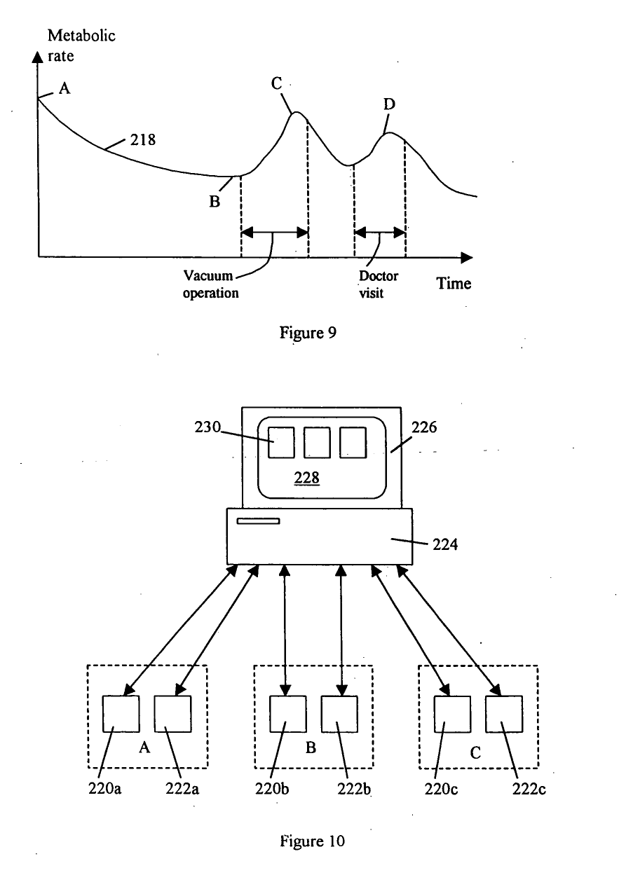

FIG. 9 illustrates a person's metabolic rate (TEE) as monitored by an indirect calorimeter over time in response to their environment. The metabolic rate is shown by solid curve 218 as a function of time, for example for a person in hospital. The value of metabolic rate is high initially at A, and then declines as the person relaxes to a lower value at B. However, due to a disturbance, the operation of vacuum cleaner in the room, the metabolic rate rises to a peak at C. After the vacuum cleaner is shut off, metabolic rate falls again as the person relaxes. However, a visit from a doctor causes metabolic rate to rise again to a peak at D. As a result of this hypothetical data, one would determine that the vacuum cleaner is a major source of stress within the hospital environment and noise reduction would be preferred to reduce the stress.

A person can be provided with an indirect calorimeter having a GPS (global positioning system) function. The person's energy expenditure can be determined as a function of the person's position. For example, to determine the energy expenditure within a work environment, the person can be tracked as they move through the work environment, and energy expenditure can be correlated with the position. The person can be provided with a weight to carry, and then the energy expenditure determined as the weight is carried around a building (such as an airport or factory). Correlation of energy expenditure with time and position can allow modifications and improvements to a workplace or other building environment, so as to reduce the energy expenditure of a person within the environment.

An indirect calorimeter can be used to quantify the stress subjected on a person by their environment. For example, a patient in a hospital (or an actor playing the role of a patient) may use the GEM to monitor TEE. Sources of stress can then be both identified and quantified in the hospital environment, e.g. bright lights, interactions with doctors, the first sight of hospital meals, etc. An indirect calorimeter can be used to quantify stress in other environments, e.g. hotels, old person's homes, apartment complexes, condominiums, retail environments, restaurants, etc., and subsequently used by architects, designers, ergonomics engineers, etc. to improve design aspects.

Energy expenditure can also be monitored during the performance of repetitive tasks, such as on a factory line. Energy expenditure can be correlated with line speed, motions of the person, and weight of components, and this can be used to determine optimum workplace conditions.

Energy expenditure can also be correlated with a workspace configuration, and used to help optimize the configuration to reduce energy output requirements.

Hence, a method for detecting and quantifying sources of disturbance and stress for a person in an environment comprises the steps of: providing an indirect calorimeter; having the person breathe through the indirect calorimeter so as to determine a metabolic rate for the person; monitoring the metabolic rate of the person as a function of time; monitoring the environment as a function of time; and correlating changes in the environment with changes in the metabolic rate of the person; whereby the effect of the environment changes on the metabolic rate of a person can be identified and measured quantitatively. The environment can be monitored using video cameras, microphones, thermometers, air quality sensors, motion sensors, or other environmental monitoring systems. The person's activity level can also be further monitored using physical activity sensors such as pedometers, other physiological sensors, or other monitor systems. Recorded events, such as noises, can be correlated with metabolic rate measurements, for example by storing metabolic rate data and other monitored data in a common database along with time provided by a clock.

Monitoring of a Subject

An attendant can assist relaxation of a subject. It can be advantageous to provide feedback to the attendant, allowing the attendant to assist the subject reach a relaxed state.

For example, a room can contain a number of subjects, each trying to achieve a relaxed state. An attendant can be present having a role of relaxation facilitator.

FIG. 10 shows a system comprising metabolic rate meters 220a, 220b and 220c; and physiological monitors 222a, 222b and 222c. A metabolic rate meter and physiological monitor are provided for each of three subjects A, B, and C. Clearly, the number of subjects can be greater; at least one subject can be monitored using a similar system configuration. Signals from the metabolic rate meters and physiological monitors are communicated to a control unit 224. The control unit can comprise a processor, memory, clock, display drives, and data receivers adapted to receive data transmitted by the metabolic rate meters and physiological monitors. A display 226 provides a visual representation 228, comprising windows 230 providing a graphical or numeric indication of the state of relaxation of each monitored subject. The window content can comprise the identity of the subject (or the window location can identify the subject), the relaxation state of the subject, the relaxation trend, a numerical display, and the like. The attendant can monitor the progress of each person towards a relaxed state using the visual representation 228. The attendant may communicate to each subject so as to assist him or her to achieve a relaxed state. For example, the attendant can be provided with a wireless microphone, and a channel selector having a channel selector switch, so as to transmit spoken advice to one or more monitored subjects. For example, each subject can be provided with an earpiece so as to receive advice, preferably transmitted by a wireless method by the attendant. The attendant can also suggest changes in the person's posture, use of scents, mantras, and the like.

Group Relaxation

A person, or group of persons, such as acolytes, may attempt to attain a Zen state or other highly relaxed state under the supervision of a master. The master may or may not have their own indirect calorimeter. The master is provided with a feedback mechanism, wherein the feedback is correlated with metabolic rate and/or physiological monitoring readings from the acolytes. Monitors or other visual indicators can be provided in front of students to indicate progress. An indirect calorimeter used by a person can include an indicator light indicating the degree of relaxation. Computer monitoring of data may be used, with feedback and advice provided using a computer expert system.

An acolyte may wear a hat, or other head-mounted device comprising a visual indication of degree of relaxation, in communication with an indirect calorimeter, so as to indicate to others, such as a master, their degree of relaxation. This system allows others to provide feedback, for example in the form of encouragement, lighting candles, repeating mantras, or other relaxing acts.

Non-Human Subjects

Embodiments of the present invention can be used to assist the relaxation of non-human subjects, hereinafter referred to as animals.

Correlation of a physiological parameter with metabolic rate for an animal can be achieved for a restrained animal, sedated animal, or partially sedated animal, if necessary. The physiological parameter can then be used to determine metabolic rate.

Feedback correlated with the animal's metabolic rate can be provided to the animal's handler, allowing the handler to take actions to assist the animal to achieve a relaxed state. This might include talking to the animal, stroking the animal, fanning the animal, and other such animal relaxing actions as appropriate.

Feedback correlated with the animal's metabolic rate may also be provided to the animal, the feedback modified according to the species of animal. For example, an animal can be rubbed using an animal-rubbing device, the rubbing speed correlated with the metabolic rate of the animal. Lights, scents, audible signals, drug administration, and the like can also be used to provide feedback to the animal.

The respiratory connector (mask, mouthpiece, helmet, or other device) is adapted according to the animal. For example, a respiratory connector for a horse can be advantageously adapted from the disclosure of U.S. Pat. No. 4,273,119 to Marchello, for example by further comprising a respiratory connector adapted to make a fluid coupling with an indirect calorimeter. The horse can be provided with feedback, for example by mechanical stroking, visual indications, audible signals, and the like. The respiratory connector can further comprise a nose-rubbing mechanism.

Hence, a method for achieving a relaxed state of a person, other mammal or other animal, comprises: providing an indirect calorimeter; having the person, other mammal, or other animal breath through the indirect calorimeter; monitoring the metabolic rate of the mammal using the indirect calorimeter; and providing a feedback mechanism so as to indicate when the metabolic rate of the mammal has fallen to a baseline level.

Self-Regulation of Metabolism

The indirect calorimeter may be used to increase fat burning. The respiratory quotient RQ is the volume ratio of carbon dioxide production to oxygen intake by the person. A high value indicates preferential metabolism of carbohydrates over fat metabolism. For carbohydrate-only metabolism, RQ is typically about 1. For fat-only metabolism, the value falls to approximately 0.7 Typically, RQ is around approximately 0.85. Indirect calorimeters such as the GEM may be used to measure RQ, using e.g. from inhalation and exhalation volume measurements, and concentration measurements of oxygen and/or carbon dioxide in the respired gases. Hence, the value of RQ may be measured as a function of time by an indirect calorimeter for a person. The person concentrates on decreasing the respiratory quotient, corresponding to an increase in fat burning. The person would sit in a relaxed state looking at the respiratory quotient meter and concentrating on decreasing the indicated RQ. The person then uses their mental abilities to increase the fat burning, as indicated by RQ. The respiratory quotient meter used to indicate the respiratory quotient to the person may take the form of a typical needle-based analog meter, digital reading, audio pitch variations, light wavelength or intensity changes, or other optical, acoustic, haptic, or aroma based feedback method. This technique may be termed biofeedback assisted fat metabolism (BAFM).

Provision of feedback correlated with metabolic rate can allow a person to control the value of their metabolic rate. An indirect calorimeter can be provided, having an inbuilt feedback mechanism or in communication with a feedback mechanism.

Feedback can also be provided to a person so as to assist the person to increase their metabolic rate, for example through tensing muscles, mental agitation, self-regulation of heart rate and/or other physiological activities, and the like.

Hence, a method to increase the fat burning proportion of metabolic processes for a person comprises: providing an indirect calorimeter; having the person breath through the indirect calorimeter; measuring the respiratory quotient of the person using the indirect calorimeter; indicating the respiratory quotient to the person; and having the person concentrate on lowering the numerical value of the respiratory quotient; whereby the person uses their mental powers to decrease the respiratory quotient indication, hence increasing the fat metabolism of the person.

Respiration Training

Embodiments of the present invention can also be used in respiration training. An indirect calorimeter is used to transmit metabolic rate, respiration frequency, and other respiratory parameters such as respiration volume, inhalation duration, exhalation duration, pause duration, and the like, to a feedback mechanism such as described elsewhere in this specification. Feedback is provided to the person correlated with metabolic rate, and also correlated with the value one or more respiratory parameters. For example, respiratory feedback can be correlated with the difference between the value of a current respiratory parameter and the desired value of a respiratory parameter. Respiratory parameters can be determined from the signals provided by a pair of ultrasonic transducers, or other flow rate sensors, within the indirect calorimeter. The provision of a first feedback correlated with metabolic rate, and a second feedback correlated with one or more respiratory parameters, is advantageous over conventional systems with regard to respiration training.

The invention is not to be restricted by specific embodiments described. Other embodiments will be clear to those skilled in the relevant arts. Having described our invention,

We claim:

1. A method for achieving a relaxed state during metabolic rate measurement, said method comprising the steps of:
breathing into an indirect calorimeter for determining oxygen consumption by the user;
determining the metabolic rate by the indirect calorimeter using the oxygen consumption;
providing the metabolic rate from the indirect calorimeter to a feedback mechanism; and
receiving feedback from the feedback mechanism by the user, correlating a relaxed state with the metabolic rate.

2. The method of claim 1, wherein the feedback correlates a difference between the metabolic rate determined for the user and a resting metabolic rate of the user.

3. The method of claim 1, wherein the feedback correlates a difference between the metabolic rate determined for the user and an initial metabolic rate determined for the user.

4. The method of claim 1, wherein the feedback is an audible signal indicating progress towards a relaxed state.

5. The method of claim 1, wherein the feedback is a visual signal indicating progress towards a relaxed state.

6. The method of claim 1, wherein the feedback is a mechanical oscillation indicating progress towards a relaxed state.

7. The method of claim 1, wherein the feedback is an aromatic signal indicating progress towards a relaxed state.

8. The method of claim 1, wherein the feedback is a synthesized speech signal indicating progress towards a relaxed state.

9. The method of claim 1, wherein the feedback is an electromagnetic radiation signal indicating progress towards a relaxed state.

10. The method of claim 1 including the step of determining if a hazardous gas is present in the environment and warning the user of the hazardous gas.

11. The method of claim 1 wherein the relaxed state is achieved when the determined metabolic rate reaches a predetermined steady state value.

12. The method of claim 1 further comprising the steps of monitoring the environment of the user over time; and correlating changes in the environment with changes in the metabolic rate of the user.

13. The method of claim 1 further including the step of monitoring an activity level for the user, and correlating activity level with changes in the metabolic rate of the user.

14. A method for achieving a relaxed state during metabolic rate measurement, said method comprising the steps of:
monitoring a physiological parameter for a user;
determining a metabolic rate for the user, wherein the metabolic rate is determined using a previously determined correlation between a physiological parameter for the user and a metabolic rate for the user; and
providing feedback to the user correlating a relaxed state with the metabolic rate determined for the user.

15. The method of claim 14 further including the steps of:
measuring a physiological parameter using a physiological monitor mounted to the user;
correlating the physiological parameter with metabolic rate; and
using the physiological parameter to monitor progress towards the predetermined relaxed state.

16. The method of claim 14, wherein the physiological parameter is a pulse rate.

17. A system for achieving a relaxed state during metabolic rate measurement of a user comprising:
an indirect calorimeter for periodically determining a metabolic rate of the user as the user breathes through the indirect calorimeter;
a feedback mechanism operatively in communication with the indirect calorimeter, wherein said feedback mechanism provides the user with feedback correlating the determined metabolic rate with a predetermined relaxed state.

18. The system of claim 17 wherein said feedback mechanism includes a display for displaying the metabolic rate.

19. The system of claim 17, wherein said feedback mechanism is a portable computing device in communication with the indirect calorimeter, wherein a the display of said portable computing device provides feedback to the user.

20. The system of claim 17, wherein the indirect calorimeter includes a mask in contact with the face of the user for transmitting the respiratory gases of the user.

21. The system of claim 17 wherein said feedback mechanism is an electronic device having a display, a data entry mechanism, a processor and an audible signaling means.

22. The system of claim 21, wherein said electronic device is a personal digital assistant.

23. The system of claim 21 wherein said electronic device is an interactive television operatively connected to an interactive television network.

24. The system of claim 21 wherein said electronic device is an entertainment device in communication with the indirect calorimeter.

25. The system of claim 15 further comprising a remote computer system in communication with said indirect calorimeter via a communications network and the remote computer system provides feedback to the user via the communications network through an entertainment device.

26. The system of claim 17 wherein the feedback is a sensation provided to the person to assist the person in achieving a relaxed state.

27. The system of claim 17 wherein the feedback is a relaxing image displayed on the display screen of a personal digital assistant.

28. The system of claim 17 wherein said indirect calorimeter includes a means for receiving a relaxing audible signal for assisting the person in achieving a relaxed state.

29. The system of claim 17 wherein said indirect calorimeter and said feedback mechanism are incorporated in a common housing.

30. The system of claim 17 further comprising a global positioning satellite receiver operatively in communication with said indirect calorimeter for determining energy expenditure of the user and correlating the energy expenditure of the user with achieving a relaxed state.

31. The system of claim 17 further comprising a plurality of metabolic rate meters operatively connected to a central control unit for monitoring the progress of a plurality of users towards a relaxed state by an attendant, and the attendant individually assisting each user in achieving a relaxed state.

32. The system of claim 17 wherein the metabolic rate is monitored by a group of persons to encourage a member of the group in achieving a relaxed state.

33. The system of claim 17 wherein the system is used to assist a non-human user in achieving a relaxed state.

34. A system of claim 17 wherein the feedback indicates a degree of relaxation.

35. A system for achieving a relaxed state during metabolic rate measurement comprising:
a support for supporting a person during metabolic rate measurement;
an indirect calorimeter for periodically determining a metabolic rate of the person as the person breathes through the indirect calorimeter;
a control device having a display screen and operatively in communication with said indirect calorimeter, wherein the determined metabolic rate is displayed on said display screen to provides the person with visual feedback correlating the determined metabolic rate with a predetermined relaxed state; and
a vibrating mechanism disposed in said support to provide the person with a physical signal indicating progress towards a relaxed state.

36. A system for achieving a relaxed state of a person during metabolic rate measurement comprising:
a physiological monitor which measures a physiological parameter that correlates with metabolic rate;
an indirect calorimeter for periodically determining a metabolic rate of the person using said measured physiological parameter while the person breathes through the indirect calorimeter; and
a feedback mechanism operatively in communication with the indirect calorimeter, wherein said feedback mechanism provides the person with feedback correlating the determined metabolic rate with a predetermined relaxed state.

37. A system as set forth in claim 36 wherein said physiological monitor is a wrist mounted device that measures the person's pulse rate.

38. A system for achieving a relaxed state during metabolic rate measurement comprising:
an indirect calorimeter for periodically determining a metabolic rate of a person while the person breathes through the indirect calorimeter;
a feedback mechanism having a controller, an environmental sensor, an environmental controller, and a display wherein said feedback mechanism is operatively in communication with the indirect calorimeter and adjusts the environmental conditions corresponding with the periodically determined metabolic rate to iteratively determine optimum environmental conditions for achieving a relaxed state, and provides the person with feedback correlating the determined metabolic rate with a predetermined relaxed state.

39. A breathing apparatus, comprising:
a source of respiratory gases;
a respiratory tube operatively connected to a respiratory connector;
an inlet tube operatively connecting said source of respiratory gases with a respiratory connector;
an indirect calorimeter operatively connected to said respiratory connector, wherein said indirect calorimeter includes a flow pathway through which exhaled and inhaled gases pass, the flow pathway having a fluid coupling with the source of inhaled gas, a sink for exhaled gas, and said respiratory connector through which the person breathes, and a respiratory gas analyzer adapted to determine the flow rate of gases and the partial pressure of at least one gas passing through at least a part of the flow pathway for determining a metabolic rate of the person;
a feedback mechanism coupled to the indirect calorimeter, and adapted to provide feedback to the person correlating metabolic rate of the person with a predetermined relaxed state.

40. The breathing apparatus of claim 39 wherein a ventilatory equivalent relates inhaled volume to oxygen consumption to determine a state of hyperventilation of the person, and corrects for carbon dioxide depletion if hyperventilation is detected.

41. The breathing apparatus of claim 39, wherein the respiratory gas analyzer is a pair of ultrasonic transducers.

42. The breathing apparatus of claim 39, wherein the respiratory gas analyzer is an oxygen sensor.

43. The breathing apparatus of claim 39, wherein the respiratory gas analyzer is a capnometer.

44. A system for achieving a relaxed state during metabolic rate measurement comprising:
a ventilator for providing respiratory gases to a person;
a metabolic rate meter operatively connected to said ventilator, wherein said metabolic rate meter is an indirect calorimeter;
a patient feeding mechanism; and
a feedback mechanism for controlling the environment of the patient to assist the patient in achieving a related state, and adjusting feeding of the patient using the patient feeding mechanism, corresponding to the metabolic rate of the patient.
